(19) United States
(12) Patent Application Publication
(10) Pub. No.: US 2020/0178892 A1
(43) Pub. Date: Jun. 11, 2020

(54) SYSTEMS AND METHODS FOR MONITORING AND MODULATING CIRCADIAN RHYTHMS

(71) Applicant: Circadia Technologies Limited, London (GB)

(72) Inventors: Michal Maslik, London (GB); Fares Siddiqui, London (GB)

(73) Assignee: Circadia Technologies Limited, London (GB)

(21) Appl. No.: 16/618,487

(22) PCT Filed: May 30, 2018
(86) PCT No.: PCT/EP2018/064306

§ 371 Date: Dec. 2, 2019

(30) Foreign Application Priority Data
May 30, 2017 (GB) 1708610.9

(51) Int. Cl. A61B 5/00; A61B 5/0205; A61M 21/02; A61N 5/06

(52) U.S. Cl. CPC A61B 5/4854; A61B 5/0205; A61B 5/4806; A61B 5/7405; A61B 5/742; A61B 5/05; A61M 21/02; A61B 5/4836; A61N 5/0618; A61B 5/0022; A61B 5/746

(57) ABSTRACT

The invention provides systems and methods for monitoring the sleep of a user and modulating a user's circadian rhythm. A monitoring device is provided for monitoring the sleep behaviour and environment of a user and a lighting device is provided for modulating the circadian rhythm of a user. In embodiments, the systems and devices comprise a motion sensor, environmental sensors and LEDs. The data 10 collected by the monitoring device allows a user's circadian rhythm to be modelled and a lighting schedule to be determined and received by the lighting device for modulating the circadian rhythm.

The present invention relates to a monitoring and lighting system and method for monitoring the sleep and modulating the circadian rhythm of a user. In embodiments, the invention relates to a monitoring device and a method for monitoring the sleep behaviour and environment of a user. In other embodiments, the invention relates to a lighting device and a method for modulating the circadian rhythm of a user.

Difficulties in falling asleep at night, getting up in the morning, getting the recommended hours of sleep each night and sleeping well enough to be fully alert during the day are common problems, particularly for teenagers and adults.

Sleeping patterns are regulated by the circadian rhythm, the roughly 24-hour cycle of many various internal biological systems. Circadian rhythms include cycles of sleeping, eating, body temperature and hormone production. Circadian rhythms are responsible for the promotion or inhibition of the release of hormones including melatonin, which causes drowsiness and puts the body in the right condition for sleep, and cortisol, the stress hormone. While circadian rhythms are affected by a number of various external cues, the factor which influences circadian rhythms the most is light.

The production of melatonin increases in the evening in preparation for sleeping, generally under dim light conditions at a point known as dim light melatonin onset (DLMO). Short wavelength light, primarily blue light, is known to be the most effective inhibitor of the production of melatonin and thus can heavily influence circadian rhythms. Exposure to blue light in the evening can delay DLMO and interrupt sleep patterns and the circadian rhythm.

For somebody experiencing sleeping problems, it can be advantageous to monitor and track patterns of sleeping stages and sleep duration in order to model their circadian rhythm. Sleep can be generally divided into rapid eye movement (REM) sleep, during which the brain is most active and muscles are paralysed, and non-rapid eye movement (NREM) sleep, which includes light sleep and deep sleep. A person tends to cycle between the sleep stages several times throughout the night. By tracking sleep patterns and combining this with other biological markers such as body temperature and actimetry, a person's circadian rhythm can be modelled. People with sleeping problems can be treated with controlled exposure to certain wavelengths of light to modulate circadian phase shifts.

US-A-2016/0015315 discloses a system to monitor and assist a user's sleep, comprising a bedside device positioned near the user's bed. The bedside device comprises a loudspeaker and a light source and optionally a microphone, a light sensor, a temperature sensor and an air quality sensor. The user's sleep is monitored by a sensing unit positioned in the user's bed which senses changes in pressure as the user moves in bed. The system can provide the user with a light program based on an assessment of the user's sleep cycles and phases. The microphone is used to detect movement, ambient noises that may disrupt a user's sleep, and irregularities in breathing that may indicate stress or sleep disorders.

US-A-2012/0209358 discloses the use of light for influencing a state of a user, including using blue light to modify melatonin levels. The blue spectrum of a light source is modified with a blue/yellow dichroic filter. A light controller of a lighting system may by controlled by an analysis engine receiving inputs regarding environmental and physiological factors. The light provided by the lighting system may consequently be adapted based upon the received factors.

WO-A-2015/006364 discloses a system for promoting sleep. The system may monitor the user's sleeping and breathing patterns and environment conditions. User sleep information, such as sleep stages and hypnograms may be recorded and evaluated. The system may further monitor ambient and/or environmental conditions corresponding to sleep sessions. Sleep advice may be generated based on the sleep information, user queries and/or environmental conditions from one or more sleep sessions. Communicated sleep advice may include content to promote good sleep habits and/or detect risky sleep conditions. The user's breathing and heart rate patterns can be monitored to allow the system to encourage the user to slow their breathing to relax and fall asleep.

US-A-2016/0158486 discloses systems and methods to provide light therapy to a subject for modifying the phase of the circadian rhythm of the subject. Light sources are configured to emit electromagnetic radiation at different intensities and parameters.

US-A-2016/0158487 discloses systems and methods to provide light therapy to a subject with pulses of substantially blue light to shift the phase of the circadian rhythm of a subject without substantially suppressing the level of melatonin production.

US-A-2016/0213309 discloses a system for determining the quality of sleep of a user by detecting the changes in body posture. The body posture of the user is determined by recording a body motion signal caused by the mechanical and muscle movements of the body of the user. Respiration and heartbeat signals can also be recorded. The respiration cycle amplitude is used to help determine the body posture of the user.

According to a first aspect of the present invention, there is provided a monitoring device for monitoring the sleep behaviour and environment of a user, wherein the monitoring device is configured to communicate with a mobile electronic device or a remote server and comprises: a motion sensor configured to detect motion of a user; one or more environmental sensors configured to measure environmental data; and a transmitter configured to transmit the motion data and environmental data to the mobile electronic device or the remote server.

The motion sensor can monitor the motion of a user while the user is asleep or awake in bed. The motion data can then be used to determine the pattern of sleep stages that the user goes through to provide information about the sleep behaviour of the user. The environmental sensors provide the advantage of being able to determine optimal sleeping conditions, or to determine the likely cause of poor quality sleep. The data collected by the environmental sensors can be correlated with the motion data or sleep patterns for the identification of, for example, a disturbance that woke the user up in the middle of the night, or an anomalous condition that persisted throughout the night that may have caused a user to wake up frequently, or only enter light sleep stages.

The data can be analysed by software on the mobile electronic device or remote server and is accessible to the user on the mobile electronic device. The motion and environmental data can therefore be used to determine how well a user is sleeping and to inform the user of ways in which they can change their environment to improve their quality of sleep.

In an example, the motion sensor is configured to detect motion caused by respiration. In one example, the motion sensor comprises a radar system and in another example, the radar system is an ultra-wideband (UWB) radar system. In a further example, the radar system is positioned at least approximately 40 centimetres above the user's chest.

Respiration can be a good indicator of whether a person is asleep or awake, and the sleep stage that they are in. Radar provides a non-contact and non-invasive way to monitor the motion and respiration, and consequently the sleep behaviour, of a user. This is much more comfortable and convenient than many current sleep monitoring systems. The user does not have to wear or sleep on any uncomfortable devices while they sleep, and the system can be easily moved to another location. UWB radar avoids interferences with other RF devices and its accuracy of UWB radar means that it can respiration patterns, as well as larger body movements, can be determined effectively.

In a further example, the motion sensor is configured to detect motion caused by heartbeats. Heart rate can be another indicator of a waking or sleeping state and the sleep stage. Monitoring heartbeats with a motion sensor, radar, or UWB radar, is more comfortable and convenient for the user as they do not have to wear any devices such as a heart rate monitor while they sleep.

In one example, the motion sensor is configured to detect individual motion of two users. This advantageously allows two people who share a bed to both monitor their sleep using a single monitoring device.

In one example, the environmental data comprise at least one of temperature, humidity, light and noise. These environmental factors can have a large impact on quality of sleep. The optimal sleeping temperature is around 18° C. and the optimal humidity is approximately in the range of 30% to 50%. Identifying how these factors correlate with sleep patterns allows a user to take action to optimise their sleeping conditions.

In one example, the monitoring device further comprises an audible alarm generator and, in another example, the monitoring device further comprises a visual alarm generator. The monitoring device can double up as an alarm clock; the sleep data allows the monitoring device to wake the user up with an alarm during a stage of light sleep. The audible alarm could be a loud noise or music; the audible alarm could be a bright light.

According to a second aspect of the present invention, there is provided a lighting device for modulating the circadian rhythm of a user, wherein the lighting device is configured to communicate with a mobile electronic device or a remote server and comprises: a plurality of LEDs; a receiver configured to receive a lighting programme from the mobile electronic device or the remote server, wherein the lighting programme contains information relating to the operation of the LEDs for modulating the circadian rhythm; and a processor configured to control the output of the plurality of LEDs in accordance with the lighting programme.

The lighting programme received by the lighting device can be determined by software on the mobile electronic device or the remote server using a model of the user's circadian rhythm. A sleep monitoring device such as that described above can provide motion data form which to determine sleep cycle data for the construction of a circadian rhythm model. Since, circadian rhythms can be influenced by light, it is possible to use the LEDs of the lighting device to output light that modulates the user's circadian rhythm.

In one example, the monitoring device further comprises the plurality of LEDs comprises at least one red LED, one blue LED and one white LED. A mixture of LED colours allows the lighting device to vary the wavelength of its light. By varying the wavelength of light, the lighting device can provide optimal colours of light to modulate the circadian rhythm.

In one example, the processor is configured to control the plurality of LEDs to output light at times, durations, colours and illuminances in accordance with the lighting programme. Different colours and illuminances have different optimal times and durations of exposure and varying these parameters can result in different effects on the circadian rhythm. This allows the light to be optimised to carry out a specific modulation of the circadian rhythm.

In one example, the lighting device further comprises a light sensor configured to detect the colour and illuminance of ambient light. In another example, the colours and illuminances of the plurality of LEDs are dependent on readings from the light sensor. The user's light exposure throughout the day can have a significant effect on their circadian rhythm, so it can be useful to record this and compare the light exposure to the circadian rhythm model. The readings from the light sensor can be used to adjust the lighting programme or the light output, if necessary, to maintain the required circadian rhythm modulation.

In one example, the plurality of LEDs is configured to output light at a corneal illuminance greater than 90 lux. Light at lower levels are less effective at influencing melatonin production and the phase of the circadian rhythm.

According to a third aspect of the present invention, there is provided a monitoring and lighting system for monitoring the sleep and modulating the circadian rhythm of a user, the system comprising: a monitoring device and a lighting device; wherein the monitoring device comprises a motion sensor configured to collect motion data for the user, and wherein the monitoring device is configured to transmit the motion data to a mobile electronic device or a remote server; wherein the motion data is used to determine sleep patterns for the user; the sleep patterns are used to determine a circadian rhythm model for the user; and the circadian rhythm model is used to determine a lighting programme to modulate the circadian rhythm of the user; and wherein the lighting device is configured to receive the lighting programme from the mobile electronic device or remote server and provide light to the user in accordance with the lighting programme.

This system provides the combined benefits of monitoring sleep behaviour and modulating the circadian rhythm. This system provides the motion data to determine sleep cycle patterns, a circadian rhythm model and a lighting programme, and the lighting device to administer the lighting programme. Therefore, the system has the advantage of being able to both provide the data for identifying sleep and circadian rhythm deficiencies, and provide the light for correcting the deficiencies. The user does not require any additional sleep monitoring or light therapy devices.

In an example, the motion sensor is configured to detect motion caused by respiration. In one example, the motion sensor comprises a radar system and in another example, the radar system is an ultra-wideband (UWB) radar system. In another example, the radar system is positioned at least approximately 40 centimetres above the user's chest. In a further example, the motion sensor is configured to detect motion caused by heartbeats. In yet another example, the motion sensor is configured to detect individual motion of two users.

In one example, the monitoring device further comprises one or more environmental sensors configured to measure environmental data. In another example, the environmental data comprise at least one of temperature, humidity, light and noise.

In one example, the monitoring device further comprises an audible alarm generator. In another example, the monitoring device further comprises a visual alarm generator.

In one example, the lighting device comprises a plurality of LEDs and a processor configured to control the output of the plurality of LEDs in accordance with the lighting programme, and in another example, the lighting programme contains information relating to the operation of the LEDs for modulating the circadian rhythm.

In one example, the plurality of LEDs comprises at least one red LED, one blue LED and one white LED. In another example, the processor is configured to control the plurality of LEDs to output light at times, durations, colours and illuminances in accordance with the lighting programme.

In one example, the lighting device further comprises a light sensor configured to detect the colour and illuminance of ambient light. In another example, the colours and illuminances of the plurality of LEDs are dependent on readings from the light sensor.

In one example, the plurality of LEDs is configured to output light at a corneal illuminance greater than 90 lux.

According to a fourth aspect of the present invention, there is provided a method for monitoring the sleep behaviour and environment of a user, the method comprising: detecting motion of a user; measuring environmental data; and transmitting the motion data and environmental data to a mobile electronic device or a remote server.

In one example, detecting motion comprises detecting motion caused by respiration. In another example, the motion is detected by a radar system. In one example, the radar system is an ultra-wideband radar system. In another example, the radar system is positioned at least approximately 40 centimetres above the user's chest.

In one example, detecting motion comprises detecting motion caused by heartbeats. In another example, detecting motion comprises detecting individual motion of two users. In a further example, the environmental data comprise at least one of temperature, humidity, light and noise.

According to a fifth aspect of the present invention, there is provided a method for modulating the circadian rhythm of a user, the method comprising: receiving a lighting programme from the mobile electronic device or the remote server, wherein the lighting programme contains information relating to the operation of a plurality of LEDs for modulating the circadian rhythm; and controlling the output of the plurality of LEDs in accordance with the lighting programme.

In one example, the plurality of LEDs comprises at least one red LED, one blue LED and one white LED. In another example, controlling the output of the plurality of LEDs comprises controlling the plurality of LEDs to output light at times, durations, colours and illuminances in accordance with the lighting programme.

In one example, the method further comprises detecting the colour and illuminance of ambient light. In another example, the colours and illuminances of the plurality of LEDs are dependent on readings from the light sensor. In a further example, the plurality of LEDs output light at a corneal illuminance greater than 90 lux.

According to a sixth aspect of the present invention, there is provided a method for monitoring the sleep and modulating the circadian rhythm of a user, the method comprising: collecting motion data for the user; transmitting the motion data to a mobile electronic device or a remote server; determining sleep patterns for the user from the motion data; determining a circadian rhythm model for the user from the sleep patterns; and determining a lighting programme from the circadian rhythm model to modulate the circadian rhythm of the user; receiving the lighting programme at a lighting device from the mobile electronic device or remote server; and providing light to the user in accordance with the lighting programme.

In one example, detecting motion comprises detecting motion caused by respiration. In another example, the motion is detected by a radar system. In one example, the radar system is an ultra-wideband radar system. In another example, the radar system is positioned at least approximately 40 centimetres above the user's chest. In one example, detecting motion comprises detecting motion caused by heartbeats. In another example, detecting motion comprises detecting individual motion of two users.

In one example, the method further comprises measuring environmental data. In another example, the environmental data comprise at least one of temperature, humidity, light and noise.

In one example, the lighting device comprises: a plurality of LEDs; and the method further comprises controlling the output of the plurality of LEDs in accordance with the lighting programme. In another example, the lighting programme contains information relating to the operation of the LEDs for modulating the circadian rhythm.

In one example, the plurality of LEDs comprises at least one red LED, one blue LED and one white LED. In another example, controlling the output of the plurality of LEDs comprises controlling the plurality of LEDs to output light at times, durations, colours and illuminances in accordance with the lighting programme. In one example, the method further comprises detecting the colour and illuminance of ambient light. In another example, the colours and illuminances of the plurality of LEDs are dependent on readings from the light sensor. In one example, the plurality of LEDs output light at a corneal illuminance greater than 90 lux.

Embodiments of the present invention will now be described in detail with reference to the accompanying drawings, in which:

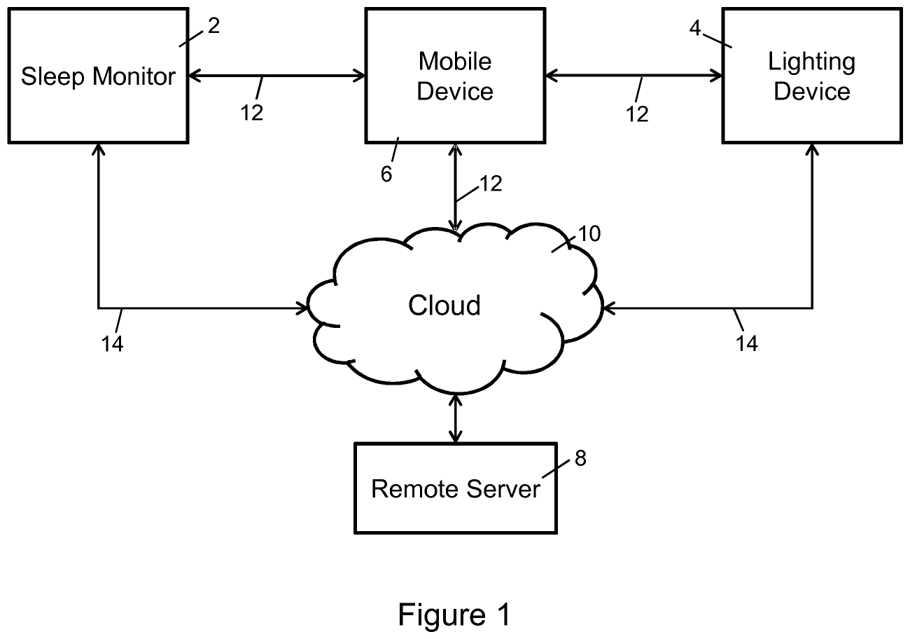

FIG. 1 is a schematic diagram of a system to monitor sleep and modulate circadian rhythms in accordance with an embodiment of the present invention;

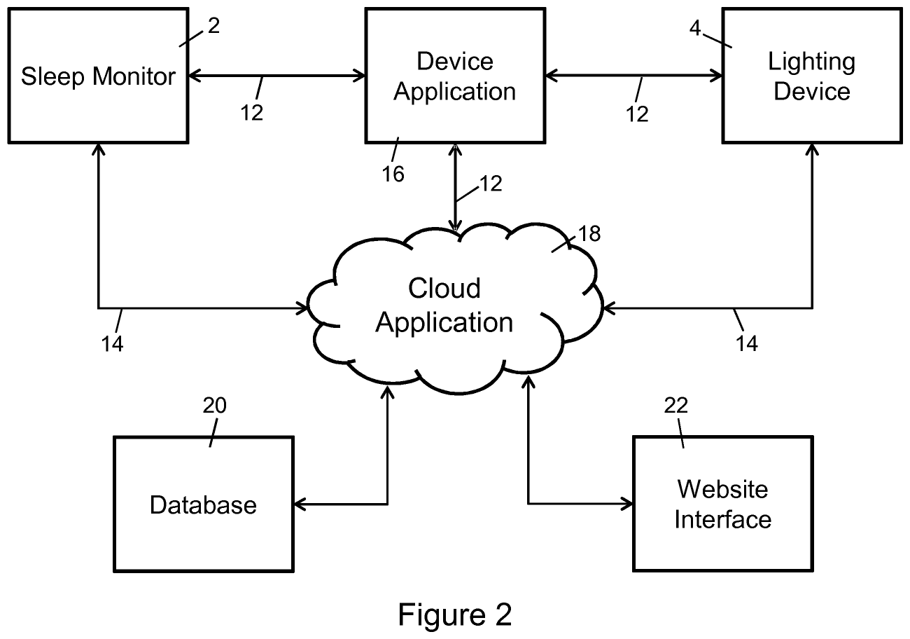

FIG. 2 is a schematic diagram of the software architecture of the system of FIG. 1;

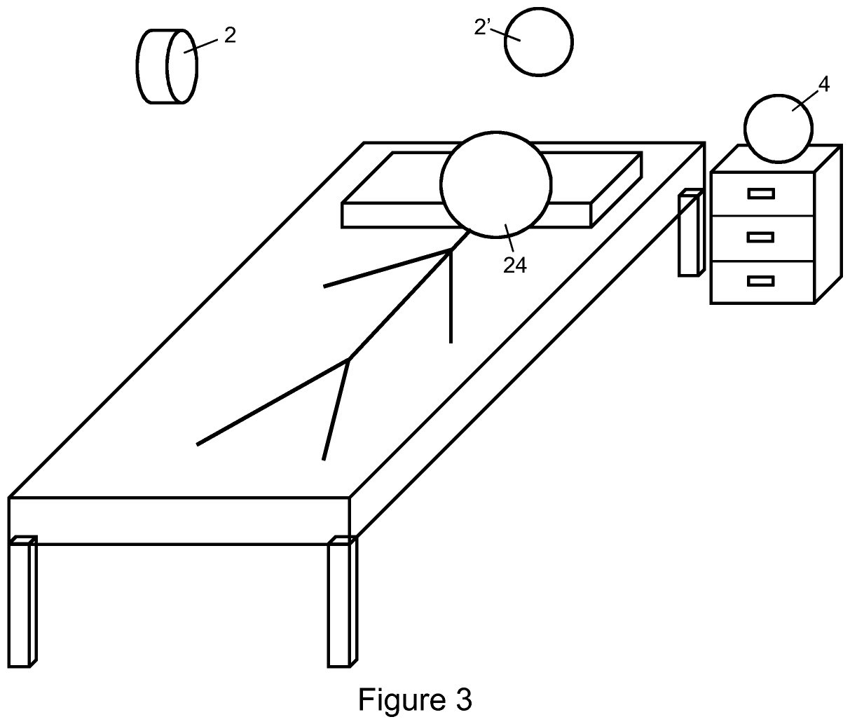

FIG. 3 shows an arrangement of sleep monitoring devices and a lighting device as a user sleeps;

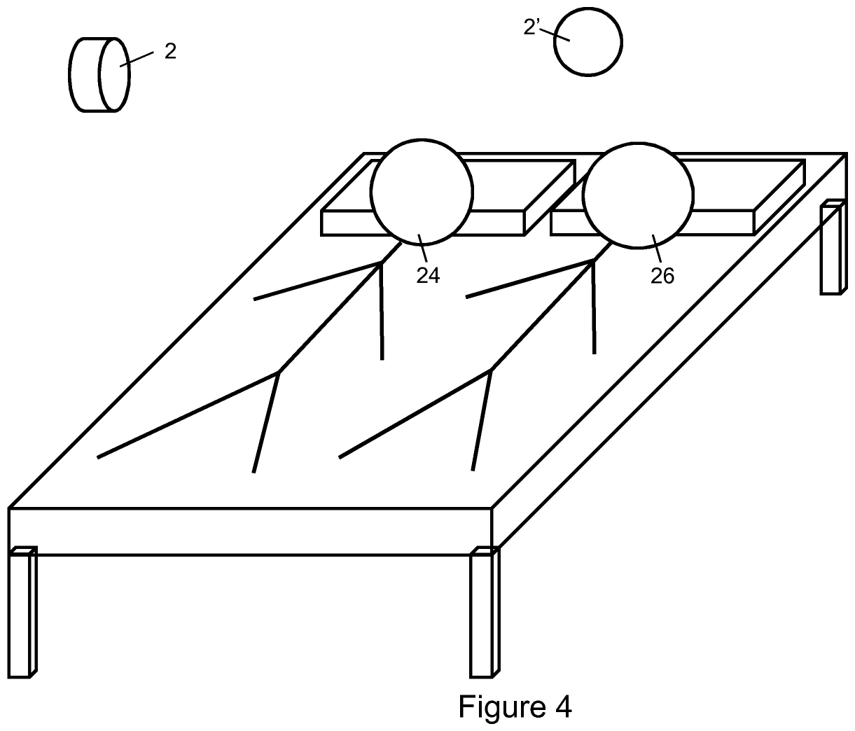

FIG. 4 shows an arrangement of sleep monitoring devices and a lighting device as two users sleep;

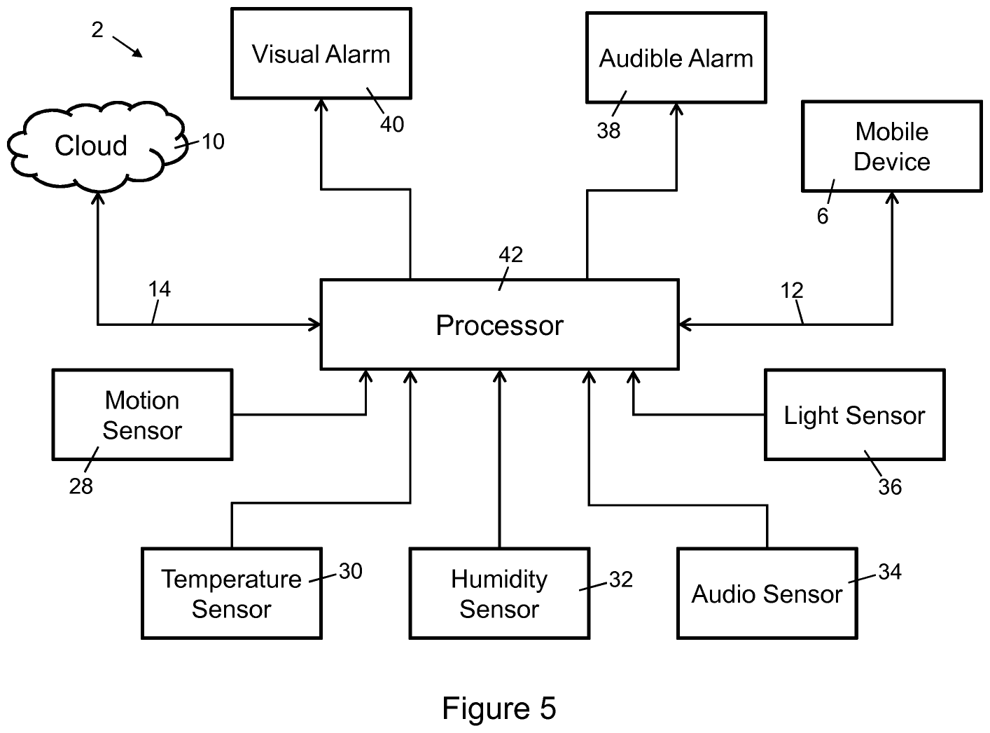

FIG. 5 is a schematic diagram of components of a sleep monitor suitable for the system of FIG. 1;

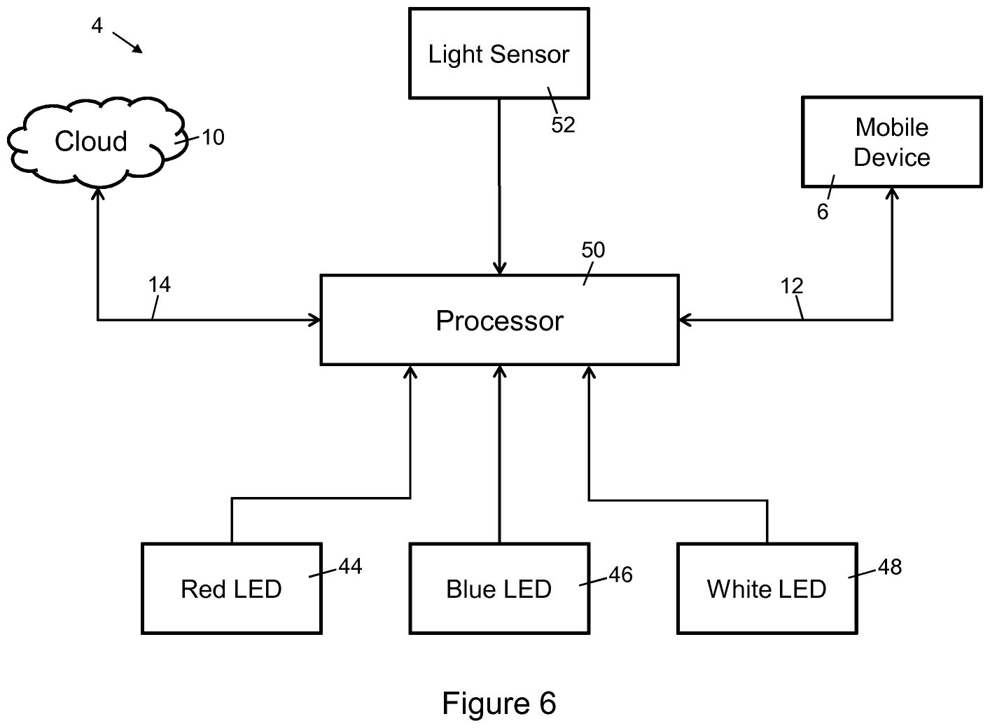

FIG. 6 is a schematic diagram of components of a lighting device suitable for the system of FIG. 1;

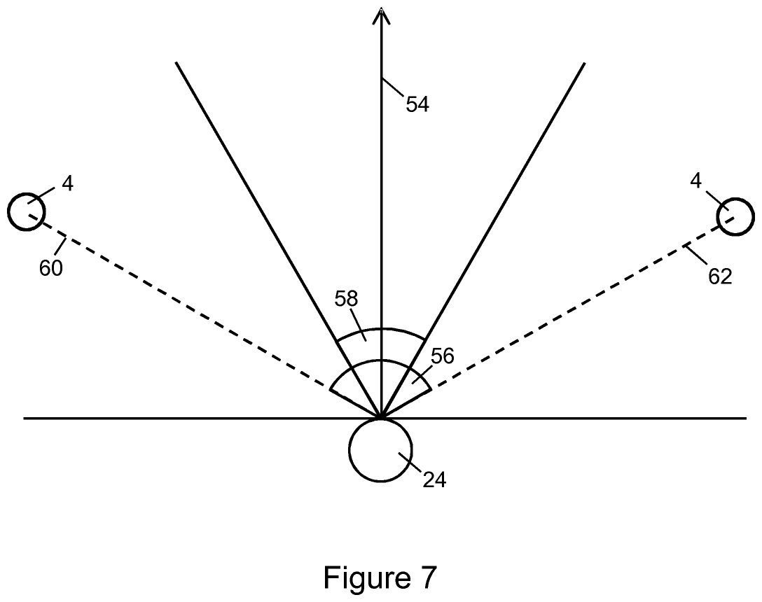

FIG. 7 shows the preferred angular positions of the lighting device; and

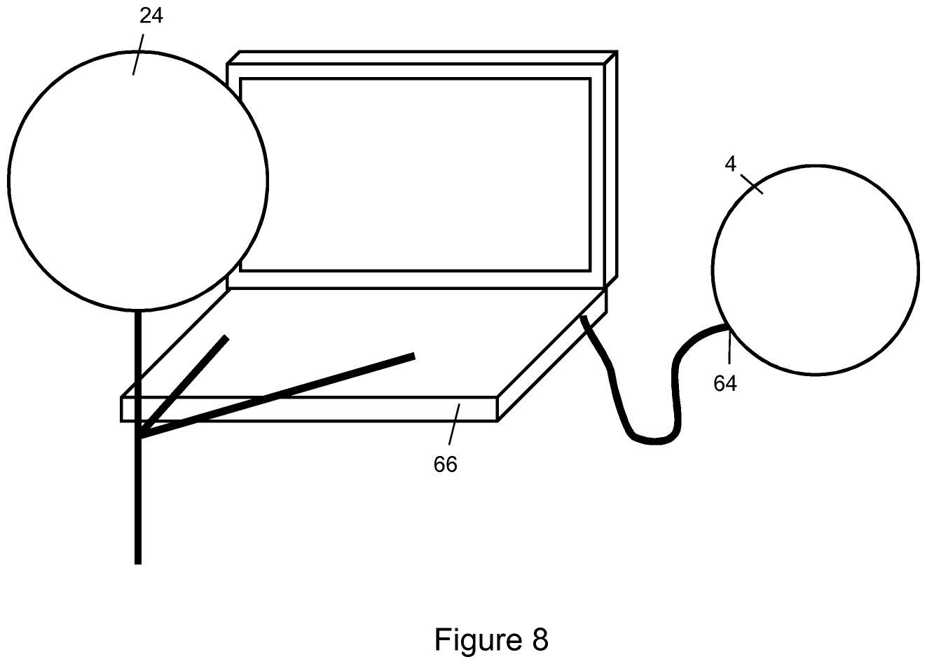

FIG. 8 demonstrates a use of the lighting device while a user works.

As illustrated in FIG. 1, a sleep monitor 2 and lighting device 4 are able to communicate with other devices, such as computing devices 6 and remote servers 8, through wired and wireless connections 12, 14 to networks such as local networks and cloud-based networks 10. In embodiments, the sleep monitor 2 and lighting device 4 can use Bluetooth connections 12 to communicate wirelessly with a computing device 6, such as through a Bluetooth low energy connection 12, and a Wi-Fi connection 14 to communicate wirelessly with a remote server 8 through a cloud-based network 10. The computing device 6 is preferably a mobile electronic device 6, such as a smartphone or tablet, but may also be a desktop PC or a laptop PC.

Referring to FIGS. 1 and 2, via the Bluetooth and Wi-Fi connections 12, 14, the sleep monitor 2 and lighting device 4 can exchange information with application software 16, 18 on the computing device 6 (device application 16) and on the remote server 8 in the cloud 10 (cloud application 18). The computing device 6 is also separately able to communicate with the cloud 10 using a Wi-Fi connection 14; through this connection 14 the device application 16 is able to exchange information with the cloud application 18. The cloud application 18 can access information held in a database 20 in the cloud 10. Information held in the database 20 may include any of sleep data collected by the sleep monitor, sleep data gathered from laboratory studies, and user information input to the device application 16. The cloud 10 also facilitates a network connection to a website interface 22 which can be accessed on any internet-connected device. The website interface 10 can provide access to data collected by the sleep monitor 2 and processed by the device application 16 or cloud application 18, for example for testing purposes.

The sleep monitor 2 monitors the sleeping behaviours and the environment of a user 24 in bed, as shown in FIGS. 3 and 4. Sleeping behaviours can include sleeping patterns, total sleep time, how long a user 24 takes to fall asleep, and whether the user 24 wakes or gets out of bed during the night. The lighting device 4 provides the user 24 with a personalised light therapy programme that has the effect of modulating the user's circadian rhythm. In embodiments, the user 24 can place the lighting device 4 on a bedside table so that the personalised light therapy programme can be administered when the user 24 wakes up or goes to bed. In some embodiments, the lighting device 4 can be located beside the bed for use as a wake-up light. However, the location of using the lighting device 4 is not limited to beside the bed or in the bedroom.

Referring now to FIG. 5, the sleep monitor 2 has at least one sensor 30, 32, 34, 36 which collects data relating to the behaviour of the user 24 and the user's environment. There are a number of environmental factors which can affect quality of sleep and it is useful to track these factors for sleep analysis. The sleep monitor 2 has a processor 42 which transmits the data collected from the environmental sensor 30, 32, 34, 36 to the computing device 6 or remote server 8 for analysis and storage. In embodiments, the sleep monitor 2 has one or both of an audible alarm generator 38 and a visual alarm generator 40 controllable by the processor 42. The user 24 can set specific times at which to be awoken by an alarm generated by the sleep monitor 2, or the user 24 can specify a window of time in which an alarm is to be generated when the user 24 is in the lightest stage of sleep.

The sleep monitor 2 has a motion sensor 28 which can detect the movements of a user's body. In embodiments, the motion sensor 28 can detect the motion of respiration, as well as larger movements of limbs or the whole body. In other embodiments, the motion sensor 28 is also able to detect motion caused by a user's heart beating.

In embodiments, the motion sensor 28 uses radar, preferably ultra-wideband (UWB) radar, to detect body movements, respiration or heartbeats. The motion sensor 28 thus provides a non-contact, non-invasive method of detecting body movements, respiration and heartbeats. Respiration and heartbeats can be detected by the movements of the chest as the lungs inflate and deflate and as the heart beats. The differing amplitudes of normal body and limb movements, respiration motion and heartbeats allow the patterns of the different types of motion to be identified from the received radar signals. The UWB radar motion sensor 28 allows recognition of several independent targets. The UWB radar motion sensor 28 therefore facilitates monitoring a plurality of people simultaneously and so the sleep monitor 2 can be used to collect data relating to the independent behaviours of two sleeping people 24, 26. This capability is advantageous since a significant proportion of adults sleep next to a partner. The sleep monitor 2 allows two people 24, 26 who sleep in the same bed to separately monitor their circadian rhythms with a single sleep monitor 2. Alternatively, multiple sleep monitors 2, 2′ can be used in a single room to monitor one or more people. For example, with reference to FIG. 3, two sleep monitors 2, 2′ can be used to monitor a single user 24. Using more than one sleep monitor 2 to monitor one user 24 provides greater accuracy in the readings. As another example, with reference to FIG. 4, a first sleep monitor 2 can be used in to monitor a first user 24, and a second sleep monitor 2′ can be used to monitor a second user 18 in the same bed as the first user 24. The XeThru Impulse Radar system by Novelda AS provides these capabilities for monitoring body movements, respiration and heartbeats using UWB radar. The UWB radar motion sensor 28 is most effective when the sleep monitor 2, 2′ is positioned within 2.5 metres of the user 24, 28. The sleep monitor 2, 2′ can be positioned in various locations, for example mounted on a wall or ceiling. The optimal height for the placement of the sleep monitor 2, 2′ is at least 40 centimetres above the chest of a user 24, 28. In embodiments, the inclination of the sleep monitor 2, 2′ can be adjusted in order to maximise the signal-to-noise ratio. In one example, the sleep monitor 2, 2′ can be tilted by up to 30° in order to allow a radar sensor with a 60° view to operate at the optimal angle of 90° to the user's chest.

By using the motion sensor 28 to monitor a sleeping person's movements, and particularly those caused by respiration, sleep cycle data such as patterns of sleeping stages and total sleep time can be determined. The frequency, depth and regularity of breathing can indicate if a person is asleep and, if so, which stage of sleeping they are in. The rate and regularity of heartbeats can also be indicative of different sleep stages. During REM sleep, breathing tends to be faster, shallower and more irregular than during NREM sleep or when a person is awake. NREM sleep can be characterised by breathing that is slower and deeper than REM and wakeful breathing. Furthermore, muscles are usually paralysed during REM sleep. Heart rates are typically slower and heartbeats are more regular in NREM sleep than REM sleep. Patterns of sleep stages can indicate the quality and duration of sleep. Correlation of the pattern of sleep stages to environmental data can help a user 24 to adjust their environment to improve their quality of sleep.

The sleep cycle can be broadly classified into four stages: two stages of NREM light sleep, one stage of NREM deep sleep, and a fourth stage of REM sleep. Analysis of movement and respiration patterns can determine the order and duration of the sleep stages, number of sleep cycles completed, and time spent awake. Additionally, the analysis can determine three key markers of sleep quality: total sleep time; sleep onset latency (time taken to fall asleep); and wake after sleep onset (WASO—time spent awake after sleep onset).

The collected motion data is transmitted by the sleep monitor 2 to a mobile electronic device 6 via a Bluetooth connection 12 or to a remote server 8 via a Wi-Fi connection 14 to a cloud-based network 10. The device application 16 can display the statistics on the mobile electronic device 6 in various ways, such as graphically or numerically, for access by the user 24. The device application 16 or cloud application 18 feeds the motion data into a sleep classification algorithm. The sleep classification algorithm analyses the motion data to identify patterns of motion and uses the patterns of motion to determine the corresponding sleep stages. In embodiments, the sleep classification algorithm is based on an autoregressive time series model alongside a deep neural network. In embodiments, the sleep classification algorithm is hosted on a virtual remote server 8 accessible through the cloud application 18.

The patterns of respiration motion can be extracted from the motion data and respiration properties such as rate and amplitude can be determined and matched to a sleep stage. In embodiments, the rate of respiration is determined and correlated with the sleep stages. The patterns of other body movements, including the frequency and amplitude of body movements, can also be extracted and used on their own, or in combination with the respiration motion patterns and properties, to determine the corresponding sleep stages. Using a combination of respiration and body motion patterns provides more accuracy in the determination of the patterns of sleep stages. For example, a period of time in which a user 24 has faster and shallower respiration but no body movement beyond the respiration motion (i.e. due to muscle paralysis) indicates that the user 24 is in REM sleep.

In embodiments, the sleep cycle data can be used by the device application 16 or cloud application 18 to determine an optimal wake-up time for the user 24, for example in a stage of light sleep. The device application 16 or cloud application 18 can use the most recently determined sleep cycle data from the most recently collected motion data to decide whether or not to send an instruction to the sleep monitor 2 to generate an audible or visual alarm in order to wake up the user 24. For example, the user 24 could use the device application 16 to input a desired window of time in which to wake up. The device application 16 or cloud application 18 can then monitor the sleep cycle data during this window of time and send the instruction to generate an alarm when the user 24 is in a stage of light sleep. If the user 24 does not enter a stage of light sleep, the alarm can be generated regardless at the end of the specified window of time.

Motion data collected by the motion sensor 28 can also be used to identify symptoms of sleeping disorders such as sleep apnoea, restless legs syndrome, and periodic limb movement disorder. Analysis of the motion data can provide separate movement patterns for different parts of the body, such as movement patterns for individual limbs, and separate respiration patterns for the thorax and the abdomen. Useful information can be gained by comparing movement patterns for different parts of the body. For example, if the respiration cycles for the thorax and abdomen are out of phase, this indicates that the user 24 is suffering from obstructive sleep apnoea. Comparing the movement patterns for individual limbs to the movement patterns for the rest of the body can provide an indication of restless legs syndrome or periodic limb movement disorder.

In embodiments, the sleep monitor 2 also has one or more environmental sensors 30, 32, 34, 36 for monitoring aspects of the environment in which the user 24 is sleeping. The environmental sensors 30, 32, 34, 36 collect environmental data to describe the sleeping conditions. Environmental data that can be collected include temperature, humidity, noise and light. The use of multiple sleep monitors 2, 2′ in one room would allow multiple readings from different locations to be combined for each type of environmental data, thus providing a more accurate snapshot of the environment of the room as a whole.

In some embodiments, the sleep monitor 2 has one or more sensors 30, 32 to monitor one or both of ambient temperature and humidity of the room in which the sleep monitor 2 is placed. The sleep monitor 2 may have a single sensor to measure both temperature and humidity, or a separate sensor 30, 32 for each. Ambient temperature and humidity are important factors to measure as they can influence quality of sleep. The discomfort caused by a bedroom being too hot or too cold or the air being too humid or too dry can make it harder to fall asleep, lower the quality of sleep, and affect the sleep cycle. Therefore it is useful to collect humidity and temperature data and correlate this with pattern of sleep stages.

In some embodiments, the sleep monitor 2 has an audio sensor 34 for monitoring noise levels while a person sleeps. Ambient noise and loud sudden noises can affect the quality of sleep and the sleep cycle. Therefore it is useful to collect noise data and correlate statistics such as volume and time of occurrence with the pattern of sleep stages.

In some embodiments, the sleep monitor 2 has a light sensor 36 to monitor the light levels while a person sleeps. A bedroom that is illuminated too brightly can make it harder to fall asleep and cause a person to wake up. This can lower quality of sleep and affect the sleep cycle. Therefore it is useful to collect light data and correlate statistics such as illuminance with the pattern of sleep stages.

Readings by the environmental sensors 30, 32, 34, 36 may be taken at regular short intervals throughout a predetermined sleeping period, or less frequently or regularly, for example just once at the beginning of each sleeping period, or at both the beginning and end of the sleeping period. If multiple environmental data readings are taken throughout one sleeping period, the readings are correlated with the sleep cycle data such as total sleep time, REM sleep time, non-REM sleep time and the pattern of sleeping stages determined using the motion data throughout the same sleeping period. Alternatively, multiple environmental data readings may be averaged to provide one data point per environmental factor per night and the environmental data averages for a series of sleeping periods are compared to the sleep cycle data for the series of sleeping periods.

The correlation of data describing environmental factors such as temperature, humidity, noise and light allows the determination of causes of poor sleep and the changes that need to be made to the environment in order to improve sleep quality. The device application 16 or cloud application 18 is enabled to correlate the environmental data with the sleep cycle data and determine relationships between them. The cloud application 18 transmits results of this analysis to the mobile electronic device 6 so that the user 24 can access the environmental data, sleep cycle data and results of the correlations on the device application 16. The results of the correlations enable a user 24 to determine their precise optimal conditions for sleeping. Alternatively, the device or cloud application 18 can analyse the results to determine the user's optimal sleeping conditions.

Understanding the relationship between sleep and environmental factors enables a user 24 to modify their environment for improved quality of sleep. The device application 16 or cloud application 18 can analyse the collected sleep and environment data and use the results of the analysis to provide recommendations that could help to improve a person's sleep cycle. Possible sleep cycle improvements can be determined by the application through several methods. One method is for the application to compare the user's sleeping conditions with standard optimal sleeping conditions that have been determined through lab-based studies, such as an optimal sleeping temperature of 18° C. Another method is for the application to identify an average or a range of the environmental conditions that were present during the sleeping periods in which the user 24 had high quality sleep. These environmental conditions can therefore be assumed to represent the optimal sleeping conditions.

Once enough data has been collected to learn the optimal sleeping conditions, for example at least one week of data, the device application 16 can advise the user 24 on the target environmental conditions for the best quality sleep and may provide tips on how the target conditions can be achieved. If the recommendations are determined by the cloud application 18, the recommendations are transmitted to the mobile electronic device 6 for access by the user 24 on the device application 16. For example, if the temperature sensor 30 detects a temperature above the determined optimal sleeping temperature range, a notification may be issued on the mobile electronic device 6, advising the user 24 of the high temperature and/or suggesting that the user 24 opens a window in order to lower the ambient temperature. In one embodiment, the mobile electronic device 6 is able to remotely control the temperature settings of a smart thermostat, such as a Nest thermostat. In preparation for sleep, the mobile electronic device 6 can adjust the temperature setting of the thermostat automatically in response to temperature readings from the temperature sensor 30 of the sleep monitor 2 and previous data collected about a user's optimal sleeping temperature.

In another example, if the sleep monitor 2 detects that low quality sleep correlates with high levels of ambient noise, a recommendation may be displayed on the mobile electronic device 6 for the user 24 to wear earplugs to bed. If the sleep monitor 2 detects that low quality sleep correlates with high levels of ambient light, a recommendation may displayed on the mobile electronic device 6 for the user 24 to wear an eye-mask to bed.

The optimal sleeping conditions and advice will become more accurate and useful the longer the sleep monitor 2 is used and collects more data. If there are limited amounts of data available, for example when the sleep monitor 2 is first being used for sleep monitoring, the device application 16 or cloud application 18 may estimate optimal conditions for the user 24 using average sleep data collected from other users of a similar demographic, or using sleep data generated in sleep laboratories.

The sleep cycle data extracted from the collected motion data by the device application 16 or cloud application 18 enable a user's circadian rhythm to be modelled. The device application 16 or cloud application 18 supplies the sleep cycle data to a circadian rhythm modelling algorithm to predict features of a user's circadian rhythm. In embodiments, the circadian rhythm modelling algorithm is hosted on a virtual remote server 8 accessible through the cloud application 18.

The algorithm can use data such as the results of laboratory studies, and the data collected by the remote server 8 from a number of sleep monitors 2 in use by a variety of users in order to produce the circadian rhythm model. Models of circadian rhythm indicators such as DLMO and core body temperature can be produced based on the sleep cycle data and used to model the circadian rhythm. Core body temperature is linked to melatonin secretion and a lower body temperature can be used as a biomarker for higher levels of melatonin. The circadian rhythm modelling algorithm is a learning algorithm, so the accuracy of the model will increase as the user 24 continues to monitor their sleeping habits with the sleep monitor 2, and as more data is collected from other users of other sleep monitors 2. The Circadian Performance Simulation Software (CPSS) from the Division of Sleep Medicine at Harvard Medical School is one example of software that could be used to produce a model of a circadian rhythm.

The resultant subjective circadian rhythm model can be used to provide the user 24 with information such as the expected variance of alertness levels throughout the day and how external factors such as jet lag and shift work are affecting their circadian rhythm. The circadian rhythm model can therefore be used to predict patterns of drowsiness, alertness and cognitive performance throughout the day, and to assist the user 24 in deciding upon changes to make to their lifestyle or daily schedule in order to optimise their performance. A circadian rhythm model can also be used to diagnose a sleep disorder, such as advanced sleep phase disorder, delayed sleep phase disorder and shift work sleep disorder.

The sleep cycle data used to create the circadian rhythm model can be supplemented with other data relating to a user's physiology and activities, such as body temperature, heart rate, and activity levels. This supplementary data may be input manually by the user 24 into the device application 16, or obtained from third-party devices such as the Apple Watch, Fitbit and Jawbone activity tracking bands. The supplementary data can be used by the circadian rhythm modelling algorithm to improve the accuracy of the model.

In order to improve the accuracy of the circadian rhythm model, the device application 16 is configured to receive input from the user 24 about their habits, activities and how they feel. The user 24 may be asked questions about caffeine and alcohol consumption, alertness and stress levels, smoking habits, exercise, mood, and diet, among other topics. The user 24 can also use the device application 16 to inform the model when a big change to their habits is about to occur, for example if the user 24 is about to go on a long-haul trip and will suffer from jet lag. These are some examples of factors which can affect the quality of sleep and influence the circadian rhythm. If the algorithm is run by the cloud application 18, the device application 16 transmits the supplementary and user-input data to the remote server 8 so that the cloud application 18 can input the data to the algorithm.

The device application 16 or cloud application 18 can correlate the user-input data with large data sets in order to make predictions based on demographic data. Large data sets may be obtained from a number of lab-based studies, surveys, or data collected by the remote server 8 from all the sleep monitors 2 that are in use by a variety of users. Correlating the user-input data with a large data set can help to provide predictions of user behaviour that has not been disclosed by the user 24, such as smoking or drinking alcohol before bed. For example, the device application 16 or cloud application 18 could note that the user's sleeping patterns correlate closely with sleeping patterns of other people who frequently smoke just before going to bed. This would suggest to the device application 16 or cloud application 18 that the user 24 does this as well and the device application 16 could use the mobile electronic device 6 to prompt the user 24 to input this information if it is correct. This helps to further improve the accuracy of the circadian rhythm model.

Tracking how alert or tired a user 24 feels throughout the day can help to identify patterns of melatonin and cortisol production and other features of the circadian rhythm, especially when correlated with larger data sets. For example, the “post-lunch dip” is a well-documented phenomenon caused by the circadian rhythm whereby many people often feel drowsy and experience a drop performance levels in the afternoon. A record of this daily drop in alertness, logged manually by the user 24, can be used by the circadian rhythm modelling algorithm to increase the accuracy of the model. The user-input data can also be used to provide additional recommendations to improve the quality of sleep, such as drinking less alcohol, or restricting caffeine consumption to between certain hours of the day.

A model of the circadian rhythm of a user 24 can indicate variations in the user's circadian rhythm in comparison to optimal models obtained from lab-based studies or other users of sleep monitors 2, or in comparison to the user's own optimal historical model that has been identified through prior use of the sleep monitor 2. Once the variations in a user's circadian rhythm has been ascertained, a personalised light therapy programme can be determined in order to modulate the circadian rhythm to reduce the variations. In embodiments, the goal of the personalised light therapy programme is to decrease sleep latency and increase total sleep time. The personalised light therapy programme is produced by the device application 16 or cloud application 18 using the circadian rhythm model and supplied to the lighting device 4 by the mobile electronic device 6 or the remote server 8. In embodiments, the personalised light therapy programme is produced by an algorithm hosted on a virtual remote server 8 accessible through the cloud application 18.

Light is one of the key influencers of circadian rhythms and can have an effect on the production of melatonin and cortisol in the human body. Light at blue wavelengths is particularly effective in entraining circadian rhythms due to its inhibitive effects on the biological production of melatonin. The most effective wavelengths in the blue spectrum are in the range of approximately 460 nanometres to 485 nanometres and the optimal wavelength is 468 nanometres. Biological melatonin production is particularly sensitive to light at 468 nanometres. In green light, 550 nanometres is the most biologically effective wavelength.

The personalised light therapy programme can indicate which colours and illuminances of light a user 24 needs at particular times and durations throughout the day in order to inhibit or promote melatonin or cortisol production to modulate the circadian rhythm to match an optimal model. For example, a circadian rhythm model may demonstrate large variations in a jet-lagged user 24. A light therapy programme based on the circadian rhythm model can help to shift the circadian rhythm to match the day/night times of the user's location in order to reduce jet lag recovery time. Another example is the use of a personalised light therapy programme to treat shift work sleep disorder by shifting a user's circadian rhythm into alignment with their wake/sleep schedule.

The personalised light therapy programme can be implemented by the lighting device 4. In the embodiment shown in FIG. 3, the lighting device 4 has at least one each of red, blue and white LEDs 44, 46, 48 and a processor 50 to control their output. In other embodiments, the lighting device 4 has a plurality of LEDs 44, 46, 48 of each colour. LEDs are advantageous over traditional incandescent light bulbs due to the small size and high efficiency of LEDs. The processor 50 is enabled to control the output of each LED colour independently in order to produce a wide range of light colours by mixing varying amounts of blue, red and white light. The lighting device 4 most effectively uses white light at a colour temperature of 5000K. The spectrum of light at 5000K has a peak around 550 nanometres, therefore it is not necessary for the lighting device 4 to use a green LED to provide this biologically effective wavelength.

The lighting device 4 is able to communicate with the mobile electronic device 6 wirelessly, for example using a Bluetooth connection 12. The lighting device 4 is also able to wirelessly communicate with a remote server 8 on a cloud-based network 10 using a Wi-Fi connection 14.

The lighting device 4 also has a light sensor 52 to measure the colour of ambient light and, in some embodiments, the illuminance of ambient light. The ambient light during waking hours may be measured by the light sensor of the lighting device 4 and transmitted to the device application 16 or cloud application 18 for analysis. The measured ambient light may be compared to the user's circadian rhythm in order to determine, for example, if the blue light levels are too high or low for the time of day. The readings of the light sensor may be used to adjust the user's personalised light therapy programme. The adjustment may be automatic, or carried out by the user 24 in response to information from the device application 16.

Light produced by the lighting device 4 is enabled to modulate circadian rhythms by varying the colour, illuminance, time of use and duration of use to match the personalised light therapy programme. The lighting device 4 may therefore produce biologically effective lighting for various functions, including preparing to sleep, waking up, working and relaxing. The light produced by the lighting device 4 can be independent of or dependent on the ambient light data collected by the light sensor 52. For example, the processor 50 of the lighting device 4 may increase the output of the blue LED 46 in order to compensate for ambient light that peaks closer to the red end of the spectrum than the blue end when the personalised light therapy programme prescribes light at blue wavelengths. The overall output of the LEDs 44, 46, 48 may also be increased by the processor 50 if the light sensor 52 detects that a room is darker than prescribed by the personalised light therapy programme. For example, if the light sensor 52 detects low levels of ambient light in the morning, the lighting device 4 may produce bluer light of a high illuminance to increase the alertness of the user 24. However, if low levels of ambient light are detected in the evening, the lighting device 4 may produce redder light of a low illuminance so as not to dazzle the eyes of the user 24 or reduce drowsiness by inhibiting melatonin production.

The luminous flux of a light source is the power emitted over visible wavelengths, taking into account the sensitivity of the human eye. The luminous fluxes of the LEDs 44, 46, 48 determine the illuminance provided by the lighting device 4. High illuminance of blue light can inhibit the production of melatonin and decrease drowsiness. The luminous flux of the blue LED 46 may be reduced to promote melatonin production, for example to prepare for bed in the evening. The red LED 44 may correspondingly be increased in luminous flux to make up for the reduction in overall luminous flux, stay at a default intermediate level, or also be reduced to provide a low level of illuminance.

Another example of varying the luminous fluxes of the LEDs 44, 46, 48 may be to increase the amount of blue light in the morning to increase the natural morning melatonin inhibition in order to feel more alert. When the luminous flux of the blue LED 46 is increased, the luminous fluxes of the other LEDs 44, 48 may also be increased to provide a higher overall illuminance, or they may be decreased to provide the same overall illuminance. Alternatively the other LEDs 44, 48 may stay at an intermediate level.

The variable lighting from the lighting device 4 can provide brighter and bluer light to inhibit melatonin production and increase alertness. This can help a user 24 wake up more naturally (by replicating daylight), reduce sleep inertia, reduce the time taken to get out of bed, boost mood, boost work productivity by increasing alertness, treat advanced sleep phase disorder and overcome the post-lunch dip. The lighting device 4 can also use dimmer and redder light to promote melatonin production and increase drowsiness. This can help a user 24 to fall asleep, reduce eye strain (associated with blue light from computer screens, for example) and treat delayed sleep phase disorder.

In embodiments, the processor 50 of the lighting device 4 can control the output of the LEDs 44, 46, 48 in response to motion detected by the motion sensor 28 of the sleep monitor 2. For example, if a user 24 is in bed with the lighting device 4 switched off and the motion sensor 28 of the sleep monitor 2 detects the movement of the user 24 sitting or standing up, the processor 50 can then cause the LEDs 44, 46, 48 to emit a dim red light that will provide the user 24 with enough illumination to perform various activities such as get up to go to the toilet or drink a glass of water. The low luminous flux and low blue light content of the dim red light minimises the extent to which the light inhibits melatonin production so that the user 24 can easily go back to sleep. If the LEDs 44, 46, 48 are not switched off while the user 24 is in bed, but are instead at a dim setting, for example as a night light, the motion sensor 28 detecting movement of the user 24 may cause the processor 50 to increase the luminous flux of the light so that the user 24 can see around the room better.

In embodiments, the lighting device 4 may also be used as a wake-up light, or visual alarm, by adjusting the output of the LEDs 44, 46, 48 so that they simulate the light of a sunrise. The processor 50 controls the LEDs 44, 46, 48 to first output light that is the colour of sunlight at a dim setting, and then gradually increase the output until the room is illuminated in light similar in colour and illuminance to a newly arisen sun. The user 24 can use the mobile application to input a desired wake-up time. An optimal wake-up time can also be determined by the device or cloud application 18 from an analysis of the sleep cycle data determined from the data collected by the sleep monitor 2. The device application 16 or cloud application 18 can use the sleep cycle data to determine when the user 24 is in a stage of light sleep, from which it is easiest to wake, and then provide the wake-up light. Waking up gradually to simulated sunlight and during a light sleep stage helps a user 24 to feel more alert in the morning. The effect of the simulated sunlight supplements the natural morning decrease in melatonin production to reduce drowsiness. The wake-up light may also or alternatively be part of the personalised light therapy programme for modulating the user's circadian rhythm. Through the communicative link 12 between the mobile electronic device 6 and the lighting device 4, the device application 16 can communicate the wake-up time to the processor 50 of the lighting device 4.

In embodiments, the variation of the output of the LEDs 44, 46, 48 by the processor 50 is dictated by the personalised light therapy programme. In addition to helping the user 24 feel more alert or drowsy at certain times, the light therapy programme causes the light from the LEDs 44, 46, 48 to modulate the user's circadian rhythm. For example, if the user 24 suffers from shift work sleep disorder, the processor 50 can control the LEDs 44, 46, 48 to produce bright light towards the blue end of the spectrum at the time when the user 24 usually wakes up and/or starts their shift. Then the processor 50 can control the LEDs 44, 46, 48 to produce dim light towards the red end of the spectrum at the time when the user 24 usually relaxes and gets ready for bed. This variation in the light produced by the LEDs 44, 46, 48 will help to modulate the user's circadian rhythm to match their wake/sleep schedule. It is particularly helpful for users who work night shifts to adjust their circadian rhythms for inhibited melatonin production during the night and non-inhibited melatonin production during the day, so that the user 24 can be alert during the night and get good quality sleep during the day.

In embodiments, the processor 50 can vary the output of the LEDs 44, 46, 48 in response to inputs from the user 24 on the device application 16. This can be as a replacement for a personalised light therapy programme, or as a temporary or permanent override of the light therapy programme. The device application 16 allows the user 24 to control and configure the lighting device 4, for example for a specific event or activity. The LEDs 44, 46, 48 can be controlled by the processor 50 to produce light of different luminous fluxes or colours in response to the user 24 selecting functions on the device application 16 that are associated with different activities such as preparing to sleep, sleeping, waking up, concentrating, relaxing, working and reading. Some default functions may be pre-programmed onto the device application 16 and other custom functions can be created and saved by the user 24. Typically, brighter light with a higher blue light content will be provided for activities which require attention or concentration, and dimmer light with a lower blue light content will be provided for events which involve relaxing or preparing to sleep.

Default or custom functions in the device application 16 can be set up with various parameters defining the desired light, including one or more of illuminance, colour and duration. Some parameters, such as duration, may be newly required each time the user 24 selects a function. For example, the user 24 may select a period of 30 minutes in which to prepare for sleeping, or 20 minutes in which to wake up and get out of bed. The application can also allow the user 24 to set up gradually changing light conditions for a custom function by specifying incrementing and decrementing behaviour of the illuminance and/or colour of the light. The device application 16 transmits the lighting programme that is defined by the function to the processor 50 of the lighting device 4 so that the lighting programme can be carried out.

In addition, the processor 50 may vary the luminous flux in response to an action by the user 24 at the lighting device 4, for example the user 24 operating a switch, pressing a button or turning a dial. In embodiments, the lighting device 4 has a dimmer switch and a colour changing switch to allow the user 24 to manually control the illuminance and colour of the light.

In embodiments, the lighting device 4 is most effective at administering the personalised light therapy programme when placed within particular distances and at particular angles to the user 24. It is preferable for the distance of the lighting device 4 from the user's eyes to be no more than approximately 1 metre. The optimum distance range is approximately 0.5 to 0.75 metres. These optimal distances ensure that the corneal illuminance of the light is sufficient to modulate the user's circadian rhythm. The lighting device 4 can compensate for further distances by increasing the illuminance of the light in order to provide at least the minimum effective corneal illuminance. When the lighting device 4 is positioned at smaller distances from the user, the illuminance of the light does not need to be so high. Using input from the user 24 about the approximate distance of the lighting device 4 into the device application 16, the lighting device 4 can receive an instruction from the mobile electronic device 6 to increase or decrease the illuminance of its light according to the distance. It is preferable that at a corneal illuminance of least 90 lux is provided to the user. Corneal illuminances below 90 lux may not be bright enough to stimulate any significant shift in circadian rhythm or inhibition of melatonin production.

FIG. 7 demonstrates the most effective angular positions in the horizontal plane with respect to the direction in which the user 24 is facing 54. It is preferable for the position of the lighting device 4 in the horizontal plane to be within a first angular range 56, and more preferably within a second angular range 58. With respect to the direction in which the user 24 is facing 54, the first angular range 56 is approximately −60° to 60°, and the second angular range 58 is approximately −45° to 45°. The first angular range 56 covers the normal range of vision for a user 24 facing straight ahead 54. It is recommended that the user 24 places the lighting device 4 at approximately either the ten o'clock position 60 or the two o'clock position 62. For the light from the lighting device 4 to be most biologically effective, the lighting device 4 should be placed within the second angular range 58. Within the second angular range 58 the amount of light incident on the user's corneas is increased, therefore the lighting device 4 is more effective and efficient at modulating the circadian rhythm. The effectiveness of the lighting device 4 can be further dependent on the inclination of its light-emitting face with respect to the surface on which it is placed. An optimal inclination is 45°. Again, this optimises the amount of light incident on the user's corneas.

In embodiments, the personalised light therapy programme is most effective at modulating the circadian rhythm when the lighting device 4 provides light for particular durations. It is preferable for the daily duration of administration of the personalised light therapy programme to be in the range of approximately 15 minutes to 8hours. The optimal exposure time can be dependent on the wavelength of light and whether melatonin secretion is being suppressed or promoted. For example, green light, particularly wavelengths of around 550 nanometres, can be most effective at suppressing melatonin secretion when the duration of exposure is less than 1.6 hours, and even 15 minutes can be enough to suppress melatonin secretion and cause a phase shift in the circadian rhythm.

In some embodiments, the sleep monitor 2 and lighting device 4 are provided as separate devices; however, in other embodiments, the sleep monitor 2 and lighting device 4 can be contained within a unitary apparatus. The sleep monitor 2 and lighting device 4 are preferably portable and can be powered by batteries or through a connection to the mains electricity supply. Preferably, the sleep monitor 2 and lighting device 4 are powered by rechargeable batteries that can be charged through a USB connection 64 to a power source, such as a laptop 66, shown in FIG. 8.

In embodiments, the portable sleep monitor 2 is sized so that it can fit easily inside an overnight bag to allow the user 24 to take the sleep monitor 2 with them when they travel, so that the user 24 does not have to miss any night of sleep monitoring while they are away from home. In embodiments, the portable lighting device 4 is sized so that it can fit easily inside a bag such as a handbag, briefcase or rucksack. This allows the user 24 to take the portable lighting device 4 wherever they go, so that the lighting device 4 can be used to administer the personalised light therapy programme throughout the day. For example, the user 24 can use the lighting device 4 to wake up in the morning, while they are getting ready and having breakfast, and then the user 24 can take the lighting device 4 with them to the office so that the personalised light therapy programme can be administered while the user 24 works. FIG. 8 demonstrates how a user 24 might have the lighting device 4 on a desk beside a laptop 66 in an office so that the lighting device 4 can administer the personalised light therapy programme while the user works.

Embodiments of the present invention have been described with particular reference to the examples illustrated. However, it will be appreciated that variations and modifications may be made to the examples described within the scope of the present invention.

1. A monitoring device for monitoring the sleep behaviour and environment of a user, wherein the monitoring device is configured to communicate with a mobile electronic device or a remote server and comprises:
a motion sensor configured to detect motion of a user;
one or more environmental sensors configured to measure environmental data; and
a transmitter configured to transmit the motion data and environmental data to the mobile electronic device or the remote server.

2. The monitoring device of claim 1, wherein the motion sensor is configured to detect motion caused by respiration.

3. The monitoring device of claim 1 or 2, wherein the motion sensor comprises a radar system.

4. The monitoring device of claim 3, wherein the radar system is an ultra-wideband radar system.

5. The monitoring device of claim 3 or 4, wherein the radar system is positioned at least approximately 40 centimetres above the user's chest.

6. The monitoring device of any preceding claim, wherein the motion sensor is configured to detect motion caused by heartbeats.

7. The monitoring device of any preceding claim, wherein the motion sensor is configured to detect individual motion of two users.

8. The monitoring device of any preceding claim, wherein the environmental data comprise at least one of temperature, humidity, light and noise.

9. The monitoring device of any preceding claim, further comprising an audible alarm generator.

10. The monitoring device of any preceding claim, further comprising a visual alarm generator.

11. A lighting device for modulating the circadian rhythm of a user, wherein the lighting device is configured to communicate with a mobile electronic device or a remote server and comprises:
a plurality of LEDs;
a receiver configured to receive a lighting programme from the mobile electronic device or the remote server, wherein the lighting programme contains information relating to the operation of the LEDs for modulating the circadian rhythm; and
a processor configured to control the output of the plurality of LEDs in accordance with the lighting programme.

12. The lighting device of claim 11, wherein the plurality of LEDs comprises at least one red LED, one blue LED and one white LED.

13. The lighting device of claim 11 or 12, wherein the processor is configured to control the plurality of LEDs to output light at times, durations, colours and illuminances in accordance with the lighting programme.

14. The lighting device of any of claims 11 to 13, further comprising a light sensor configured to detect the colour and illuminance of ambient light.

15. The lighting device of claim 14, wherein the colours and illuminances of the plurality of LEDs are dependent on readings from the light sensor.

16. The lighting device of any of claims 11 to 15, wherein the plurality of LEDs is configured to output light at a corneal illuminance greater than 90 lux.

17. A monitoring and lighting system for monitoring the sleep and modulating the circadian rhythm of a user, the system comprising a monitoring device and a lighting device;
wherein the monitoring device comprises a motion sensor configured to collect motion data for the user, and wherein the monitoring device is configured to transmit the motion data to a mobile electronic device or a remote server;
wherein the motion data is used to determine sleep patterns for the user; the sleep patterns are used to determine a circadian rhythm model for the user; and the circadian rhythm model is used to determine a lighting programme to modulate the circadian rhythm of the user; and
wherein the lighting device is configured to receive the lighting programme from the mobile electronic device or remote server and provide light to the user in accordance with the lighting programme.

18. The system of claim 17, wherein the motion sensor is configured to detect motion caused by respiration.

19. The system of claim 17 or 18, wherein the motion sensor comprises a radar system.

20. The system of any of claims 17 to 19, wherein the radar system is an ultra-wideband radar system.

21. The system of claim 19 or 20, wherein the radar system is positioned at least approximately 40 centimetres above the user's chest.

22. The system of any of claims 17 to 21, wherein the motion sensor is configured to detect motion caused by heartbeats.

23. The system of any of claims 17 to 22, wherein the motion sensor is configured to detect individual motion of two users.

24. The system of any of claims 17 to 23, wherein the monitoring device further comprises one or more environmental sensors configured to measure environmental data.

25. The system of claim 24, wherein the environmental data comprise at least one of temperature, humidity, light and noise.

26. The system of any of claims 17 to 25, wherein the monitoring device further comprises an audible alarm generator.

27. The system of any of claims 17 to 26, wherein the monitoring device further comprises a visual alarm generator.

28. The system of any of claims 17 to 27, wherein the lighting device comprises:
a plurality of LEDs; and
a processor configured to control the output of the plurality of LEDs in accordance with the lighting programme.

29. The system of claim 28, wherein the lighting programme contains information relating to the operation of the LEDs for modulating the circadian rhythm.

30. The system of claim 28 or 29 wherein the plurality of LEDs comprises at least one red LED, one blue LED and one white LED.

31. The system of any of claims 28 to 30, wherein the processor is configured to control the plurality of LEDs to output light at times, durations, colours and illuminances in accordance with the lighting programme.

32. The system of any of claims 28 to 31, wherein the lighting device further comprises a light sensor configured to detect the colour and illuminance of ambient light.

33. The system of claim 32, wherein the colours and illuminances of the plurality of LEDs are dependent on readings from the light sensor.

34. The system of any of claims 28 to 33, wherein the plurality of LEDs is configured to output light at a corneal illuminance greater than 90 lux.

35. A method for monitoring the sleep behaviour and environment of a user, the method comprising:
detecting motion of a user;
measuring environmental data; and
transmitting the motion data and environmental data to a mobile electronic device or a remote server.

36. The method of claim 35, wherein detecting motion comprises detecting motion caused by respiration.

37. The method of claim 35 or 36, wherein the motion is detected by a radar system.

38. The method of claim 37, wherein the radar system is an ultra-wideband radar system.

39. The method of claim 37 or 38, wherein the radar system is positioned at least approximately 40 centimetres above the user's chest.

40. The method of any of claims 35 to 39, wherein detecting motion comprises detecting motion caused by heartbeats.

41. The method of any of claims 35 to 40, wherein detecting motion comprises detecting individual motion of two users.

42. The method of any of claims 35 to 41, wherein the environmental data comprise at least one of temperature, humidity, light and noise.

43. A method for modulating the circadian rhythm of a user, the method comprising:
receiving a lighting programme from the mobile electronic device or the remote server, wherein the lighting programme contains information relating to the operation of a plurality of LEDs for modulating the circadian rhythm; and
controlling the output of the plurality of LEDs in accordance with the lighting programme.

44. The method of claim 43, wherein the plurality of LEDs comprises at least one red LED, one blue LED and one white LED.

45. The method of claim 43 or 44, wherein controlling the output of the plurality of LEDs comprises controlling the plurality of LEDs to output light at times, durations, colours and illuminances in accordance with the lighting programme.

46. The method of any of claims 43 to 45, further comprising detecting the colour and illuminance of ambient light.

47. The method of claim 46, wherein the colours and illuminances of the plurality of LEDs are dependent on readings from the light sensor.

48. The method of any of claims 43 to 47, wherein the plurality of LEDs output light at a corneal illuminance greater than 90 lux.

49. A method for monitoring the sleep and modulating the circadian rhythm of a user, the method comprising:
collecting motion data for the user;
transmitting the motion data to a mobile electronic device or a remote server;
determining sleep patterns for the user from the motion data;
determining a circadian rhythm model for the user from the sleep patterns; and
determining a lighting programme from the circadian rhythm model to modulate the circadian rhythm of the user;
receiving the lighting programme at a lighting device from the mobile electronic device or remote server; and
providing light to the user in accordance with the lighting programme.

50. The method of claim 49, wherein detecting motion comprises detecting motion caused by respiration.

51. The method of claim 49 or 50, wherein the motion is detected by a radar system.

52. The method of claim 51, wherein the radar system is an ultra-wideband radar system.

53. The method of claim 51 or 52, wherein the radar system is positioned at least approximately 40 centimetres above the user's chest.

54. The method of any of claims 49 to 53, wherein detecting motion comprises detecting motion caused by heartbeats.

55. The method of any of claims 49 to 54, wherein detecting motion comprises detecting individual motion of two users.

56. The method of any of claims 49 to 55, further comprising measuring environmental data.

57. The method of claim 56, wherein the environmental data comprise at least one of temperature, humidity, light and noise.

58. The method of any of claims 49 to 57, wherein the lighting device comprises:
a plurality of LEDs; and
the method further comprises controlling the output of the plurality of LEDs in accordance with the lighting programme.

59. The method of claim 58, wherein the lighting programme contains information relating to the operation of the LEDs for modulating the circadian rhythm.

60. The method of claim 58 or 59, wherein the plurality of LEDs comprises at least one red LED, one blue LED and one white LED.

61. The method of any of claims 58 to 60, wherein controlling the output of the plurality of LEDs comprises controlling the plurality of LEDs to output light at times, durations, colours and illuminances in accordance with the lighting programme.

62. The method of any of claims 58 to 61, further comprising detecting the colour and illuminance of ambient light.

63. The method of claim 62, wherein the colours and illuminances of the plurality of LEDs are dependent on readings from the light sensor.

64. The method of any of claims 58 to 63, wherein the plurality of LEDs output light at a corneal illuminance greater than 90 lux.
